SEQUENCE LISTING (6 sequences)

SEQ ID NO: 1 - DNA, 3493 bp, Homo sapiens
  CDS (32)..(1714)
agttactttggagtgcagaaccatttcagacatgctgagggggactctactgtgcgcggtgctcgggcttctgcgcgcccagcccttcccctgtccgccagcttgcaagtgtgtcttccgggacgccgcgcagtgctcggggggcgacgtggcgcgcatctccgcgctaggcctgcccaccaacctcacgcacatcctgctcttcggaatgggccgcggcgtcctgcagagccagagcttcagcggcatgaccgtcctgcagcgcctcatgatctccgacagccacatttccgccgttgcccccggcaccttcagtgacctgataaaactgaaaaccctgaggctgtcgcgcaacaaaatcacgcatcttccaggtgcgctgctggataagatggtgctcctggagcagttgtttttggaccacaatgcgctaaggggcattgaccaaaacatgtttcagaaactggttaacctgcaggagctcgctctgaaccagaatcagctcgatttccttcctgccagtctcttcacgaatctggagaacctgaagttgttggatttatcgggaaacaacctgacccacctgcccaaggggttgcttggagcacaggctaagctcgagagacttctgctccactcgaaccgccttgtgtctctggattcggggctgttgaacagcctgggcgccctgacggagctgcagttccaccgaaatcacatccgttccatcgcacccggggccttcgaccggctcccaaacctcagttctttgacgctttcgagaaaccaccttgcgtttctcccctctgcgctctttcttcattcgcacaatctgactctgttgactctgttcgagaacccgctggcagagctcccgggggtgctcttcggggagatggggggcctgcaggagctgtggctgaaccgcacccagctgcgcaccctgcccgccgccgccttccgcaacctgagccgcctgcggtacttaggggtgactctgagcccgcggctgagcgcgcttccgcagggcgccttccagggccttggcgagctccaggtgctcgccctgcactccaacggcctgaccgccctccccgacggcttgctgcgcggcctcggcaagctgcgccaggtgtccctgcgccgcaacaggctgcgcgccctgccccgtgccctcttccgcaatctcagcagcctggagagcgtccagctcgaccacaaccagctggagaccctgcctggcgacgtgtttggggctctgccccggctgacggaggtcctgttggggcacaactcctggcgctgcgactgtggcctggggcccttcctggggtggctgcggcagcacctaggcctcgtgggcggggaagagcccccacggtgcgcaggccctggggcgcacgccggcctgccgctctgggccctgccggggggtgacgcggagtgcccgggcccccggggcccgcctccccgccccgctgcggacagctcctcggaagcccctgtccacccagccttggctcccaacagctcagaaccctgggtgtgggcccagccggtgaccacgggcaaaggtcaagatcatagtccgttctgggggttttattttctgcttttagctgttcaggccatgatcaccgtgatcatcgtgtttgctatgattaaaattggccaactctttcgaaaattaatcagagagagagcccttgggtaaaccaatgggaaaatcttctaattacttagaacctgaccagatgtggctcggaggggaatccagacccgctgctgtcttgctctccctcccctccccactcctcctctcttcttcctcttctctctcactgccacgccttcctttccctcctcctccccctctccgctctgtgctcttcattctcacaggcccgcaacccctcctctctgtgtcccccgcccgttcctggaaactgagcttgacgtttgtaaactgtggttgcctgccttccccagctcccacgcgggtgtgcgctgacactgccgggggcgctggactgtgttggacccatccgtgctccgctgtgcctggcttggcgtctggtggagagaggggcctcttcagtgtctactgagtaaggggacagctccaggccggggcctgtctcctgcacagagtaagccggtaaatgtttgtgaaatcaatgcgtggataaaggaacacatgccatccaagtgatgatggcttttcctggagggaaaggataggctgttgctctatctaattttttgtttttgtttttggacagtctagctctgtggcccaggctggcgtgcagtgggccgtctcagttcactgcagcctccgcctcccaggttcaagtgattctcatgcctcagcgttctgagtagctgggattagaggcgtgtgccactacacccggctaatttttgtactttttaaagtagagacggggctttgccatattggcctggctgatctcaaactcctggtcttgaactcctggccacaagtgatctgcccgccttggcctcccaaagtgctgggattacaggcgtaagccactacacctggccctcttcatcgaattttatttgagaagtagagctcttgccattttttcccttgctccatttttctcactttatgtctctctgacctatgggctacttgggagagcactggactccattcatgcatgagcattttcaggataagcgacttctgtgaggctgagagaggaagaaaacacggagccttccctccaggtgcccagtgtaggtccagcgtgtttcctgagcctcctgtgagtttccacttgctttacatccatgcaacatgtcattttgaaactggattgatttgcatttcctggaactctgccacctcatttcacaagcatttatggagcagttaacatgtgactggtattcatgaatataatgataagcttgattctagttcagctgctgtcacagtctcatttgttcttccaactgaaagccgtaaaacctttgttgctttaattgaatgtctgtgcttatgagaggcagtggttaaaacaggggctggcgagttgacaactgtgggttcaaatcccagctctaccacttactaactgcatgggactttgggtaagacacctgcttacattctctaagccttggtttcctgaaccttaaaacaggataacatagtacctgcttcgtagagtttttgtgagaattaaaggcaataaagcatataatgacttagcccagcggcctgcaggcaatacatgttaatgaatgttagctattattactaaaggatgagcaattattattggcatcatgatttctaaagaagagctttgagttggtatttttctctgtgtataagggtaagtccgaactttctcagactggaggttacattcacatcagtctgtcttcccctgcggatggcctcagccctgggtggccagactctgtgctcacaatccagagcaatggatcc

SEQ ID NO: 2 - protein, 560 aa, Homo sapiens
MLRGTLLCAVLGLLRAQPFPCPPACKCVFRDAAQCSGGDVARISALGLPTNLTHILLFGMGRGVLQSQSFSGMTVLQRLMISDSHISAVAPGTFSDLIKLKTLRLSRNKITHLPGALLDKMVLLEQLFLDHNALRGIDQNMFQKLVNLQELALNQNQLDFLPASLFTNLENLKLLDLSGNNLTHLPKGLLGAQAKLERLLLHSNRLVSLDSGLLNSLGALTELQFHRNHIRSIAPGAFDRLPNLSSLTLSRNHLAFLPSALFLHSHNLTLLTLFENPLAELPGVLFGEMGGLQELWLNRTQLRTLPAAAFRNLSRLRYLGVTLSPRLSALPQGAFQGLGELQVLALHSNGLTALPDGLLRGLGKLRQVSLRRNRLRALPRALFRNLSSLESVQLDHNQLETLPGDVFGALPRLTEVLLGHNSWRCDCGLGPFLGWLRQHLGLVGGEEPPRCAGPGAHAGLPLWALPGGDAECPGPRGPPPRPAADSSSEAPVHPALAPNSSEPWVWAQPVTTGKGQDHSPFWGFYFLLLAVQAMITVIIVFAMIKIGQLFRKLIRERALG

SEQ ID NO: 3 - protein, 544 aa, Homo sapiens
QPFPCPPACKCVFRDAAQCSGGDVARISALGLPTNLTHILLFGMGRGVLQSQSFSGMTVLQRLMISDSHISAVAPGTFSDLIKLKTLRLSRNKITHLPGALLDKMVLLEQLFLDHNALRGIDQNMFQKLVNLQELALNQNQLDFLPASLFTNLENLKLLDLSGNNLTHLPKGLLGAQAKLERLLLHSNRLVSLDSGLLNSLGALTELQFHRNHIRSIAPGAFDRLPNLSSLTLSRNHLAFLPSALFLHSHNLTLLTLFENPLAELPGVLFGEMGGLQELWLNRTQLRTLPAAAFRNLSRLRYLGVTLSPRLSALPQGAFQGLGELQVLALHSNGLTALPDGLLRGLGKLRQVSLRRNRLRALPRALFRNLSSLESVQLDHNQLETLPGDVFGALPRLTEVLLGHNSWRCDCGLGPFLGWLRQHLGLVGGEEPPRCAGPGAHAGLPLWALPGGDAECPGPRGPPPRPAADSSSEAPVHPALAPNSSEPWVWAQPVTTGKGQDHSPFWGFYFLLLAVQAMITVIIVFAMIKIGQLFRKLIRERALG

SEQ ID NO: 4 - DNA, 2215 bp, Mus musculus
  CDS (30)..(1733)
tcagtccagggtgcagaactgcttcagacatgctaagaagcgccctgctgtccgcggtgctcgcactcttgcgtgcccaaccttttccctgccccaaaacctgcaagtgtgtggtccgcgatgccgcgcagtgctcgggcggcagcgtggctcacatcgctgagctaggtctgcctacgaacctcacacacatcctgctcttccgaatggaccagggcatattgcggaaccacagcttcagcggcatgacagtccttcagcgcctgatgctctcagatagccacatttccgccatcgaccccggcaccttcaatgacctggtaaaactgaaaaccctcaggttgacgcgcaacaaaatctctcgtcttccacgtgcgatcctggataagatggtactcttggaacagctgttcttggaccacaatgcactaagggaccttgatcaaaacctgtttcagcaactgcgtaaccttcaggagctcggtttgaaccagaatcagctctcttttcttcctgctaaccttttctcgagcctgagagaactgaagttgttggatttatcgcgaaacaacctgacccacctgcccaagggactgcttggggctcaagttaagcttgagaaactgctgctctattcaaaccagctcacgtctgtggattcggggctgctgagcaacctgggcgccctgactgagctgcggctggagcggaatcacctccgctccgtagccccgggtgccttcgaccgcctcggaaacctgagctccttgactctatccggaaacctcctggagtctctgccgcccgcgctcttccttcacgtgagcagcgtgtctcggctgactctgttcgagaaccccctggaggagctcccggacgtgttgttcggggagatggccggcctgcgggagctgtggctgaacggcacccacctgagcacgctgcccgccgctgccttccgcaacctgagcggcttgcagacgctggggctgacgcggaacccgcgcctgagcgcgctcccgcgcggcgtgttccagggcctacgggagctgcgcgtgctcgcgctgcacaccaacgccctggcggagctgcgggacgacgcgctgcgcggcctcgggcacctgcgccaggtgtcgctgcgccacaaccggctgcgggccctgccccgcacgctcttccgcaacctcagcagcctcgagagcgtgcagctagagcacaaccagctggagacgctgccaggagacgtgttcgcggctctgccccagctgacccaggtcctgctgggtcacaacccctggctctgcgactgtggcctgtggcccttcctccagtggctgcggcatcacccggacatcctgggccgagacgagcccccgcagtgccgtggcccggagccacgcgccagcctgtcgttctgggagctgctgcagggtgacccgtggtgcccggatcctcgcagcctgcctctcgaccctccaaccgaaaatgctctggaagccccggttccgtcctggctgcctaacagctggcagtcccagacgtgggcccagctggtggccaggggtgaaagtcccaataacaggctctactggggtctttatattctgcttctagtagcccaggccatcatagccgcgttcatcgtgtttgccatgattaaaatcggccagctgtttcgaacattaatcagagagaagctcttgttagaggcaatgggaaaatcgtgtaactaatgaaactgaccagagcattgtggacggggccccaaggagaatgcagtcaggatgctggcgtgccattacactatttcccaggccttttctcctctcccgtgctcttagtgtctcttcttctcccctctcttcagaagtagcttttgtaaatcgctactgctttctagcctggcctgggttacctcctctgctgttagtttcaagggggctgagggtgggggttcgacgggacttggctcatcaggtccaactgtgcagcgctgggtgcctagtggagagaggagccctttcttggtttctgaatttgaggacacatcctgccagtgggcaagacctctccgggacccagcaagggttgagtaacatttgctgaaggaacaccggcttaaaacgaaccctaggtccaagagatgaaggctcttcccaaaataaaggtggagtgttcttgtccctttacctgaaaggaaaaaaaaaaaaaaaaa

SEQ ID NO: 5 - protein, 567 aa, Mus musculus
MLRSALLSAVLALLRAQPFPCPKTCKCVVRDAAQCSGGSVAHIAELGLPTNLTHILLFRMDQGILRNHSFSGMTVLQRLMLSDSHISAIDPGTFNDLVKLKTLRLTRNKISRLPRAILDKMVLLEQLFLDHNALRDLDQNLFQQLRNLQELGLNQNQLSFLPANLFSSLRELKLLDLSRNNLTHLPKGLLGAQVKLEKLLLYSNQLTSVDSGLLSNLGALTELRLERNHLRSVAPGAFDRLGNLSSLTLSGNLLESLPPALFLHVSSVSRLTLFENPLEELPDVLFGEMAGLRELWLNGTHLSTLPAAAFRNLSGLQTLGLTRNPRLSALPRGVFQGLRELRVLALHTNALAELRDDALRGLGHLRQVSLRHNRLRALPRTLFRNLSSLESVQLEHNQLETLPGDVFAALPQLTQVLLGHNPWLCDCGLWPFLQWLRHHPDILGRDEPPQCRGPEPRASLSFWELLQGDPWCPDPRSLPLDPPTENALEAPVPSWLPNSWQSQTWAQLVARGESPNNRLYWGLYILLLVAQAIIAAFIVFAMIKIGQLFRTLIREKLLLEAMGKSCN

SEQ ID NO: 6 - protein, 551 aa, Mus musculus
QPFPCPKTCKCVVRDAAQCSGGSVAHIAELGLPTNLTHILLFRMDQGILRNHSFSGMTVLQRLMLSDSHISAIDPGTFNDLVKLKTLRLTRNKISRLPRAILDKMVLLEQLFLDHNALRDLDQNLFQQLRNLQELGLNQNQLSFLPANLFSSLRELKLLDLSRNNLTHLPKGLLGAQVKLEKLLLYSNQLTSVDSGLLSNLGALTELRLERNHLRSVAPGAFDRLGNLSSLTLSGNLLESLPPALFLHVSSVSRLTLFENPLEELPDVLFGEMAGLRELWLNGTHLSTLPAAAFRNLSGLQTLGLTRNPRLSALPRGVFQGLRELRVLALHTNALAELRDDALRGLGHLRQVSLRHNRLRALPRTLFRNLSSLESVQLEHNQLETLPGDVFAALPQLTQVLLGHNPWLCDCGLWPFLQWLRHHPDILGRDEPPQCRGPEPRASLSFWELLQGDPWCPDPRSLPLDPPTENALEAPVPSWLPNSWQSQTWAQLVARGESPNNRLYWGLYILLLVAQAIIAAFIVFAMIKIGQLFRTLIREKLLLEAMGKSCN

(19) United States
(12) Patent Application Publication
(10) Pub. No.: US 2019/0169307 A1
(43) Pub. Date: Jun. 6, 2019

(54) GLYCOPROTEIN V INHIBITORS FOR USE AS COAGULANTS

(71) Applicant: JULIUS-MAXIMILIANS-UNIVERSITÄT WÜRZBURG, Würzburg (DE)

(72) Inventors: Bernhard NIESWANDT, Eibelstadt (DE); Sarah BECK, Würzburg (DE); David STEGNER, Würzburg (DE)

(21) Appl. No.: 16/065,300

(22) PCT Filed: Dec. 23, 2016
(86) PCT No.: PCT/EP2016/082572

§ 371 Date: Jun. 22, 2018

(30) Foreign Application Priority Data
Dec. 23, 2015 (EP) 15202582.1

(51) Int. Cl. C07K 16/28; A61P 7/04; C12N 15/113; A61K 39/395; A61K 45/06; A61K 31/7088

(52) U.S. Cl. CPC C07K 16/2896; A61P 7/04; C12N 15/1138; A61K 39/3955; A61K 2039/505; A61K 31/7088; C07K 2317/32; C07K 2317/92; C07K 2317/55; A61K 45/06

(57) ABSTRACT

The present invention relates to an inhibitor of platelet glycoprotein V (GPV) for use as a coagulant, and/or for use in the treatment or prevention of a hemorrhagic condition.

### BACKGROUND

Platelet activation and subsequent thrombus formation at sites of vascular injury is crucial for normal hemostasis, but it can also cause myocardial infarction and stroke (Coughlin S R. Nature. 2000; 407: 258-64). Platelet adhesion and activation is a multistep process involving multiple platelet receptor-ligand interactions. Upon vessel wall injury, circulating platelets are rapidly decelerated by transient interactions of the glycoprotein (GP) Ib-V-IX complex with von Willebrand factor (vWF) immobilized on the exposed subendothelial extracellular matrix (e.g. on collagen) (Shapiro M J, et al. JBC. 2000; 275: 25216-21). This interaction retains platelets close to the vessel wall and facilitates the contact between GPVI and collagen (Nieswandt B, et al. Blood. 2003; 102: 449-61.). GPVI-collagen interactions induce an intracellular signaling cascade leading to platelet activation and the release of secondary platelet agonists, such as thromboxane A₂ (TxA₂) and adenosine diphosphate (ADP). These soluble agonists together with locally produced thrombin further contribute to platelet activation through G protein (Gᵢ, G_{q}, G_{12/13}) coupled receptors (Offermanns S. Circulation research. 2006; 99: 1293-304). All these signaling pathways synergize to induce complex cellular responses, such as activation of integrins, release of granule contents and the provision of a pro-coagulant surface for the activation of the coagulation cascade (Nakanishi-Matsui M, et al. Nature. 2000; 404: 609-13; Cunningham M A, et al. J Exp Med 2000; 191: 455-62). The final thrombus is embedded in a fibrin network to withstand the shear forces generated by the flowing blood. The stabilization of a newly formed thrombus is essential to arrest bleeding at sites of vascular injury.

Their central role in platelet adhesion puts two receptor complexes in the focus of platelet research: i) the GPIb-V-IX complex which interacts with vWF immobilized on the injured vessel wall or on activated platelets and thereby recruits platelets from the blood stream to the reactive surface under conditions of elevated shear. ii) GPIIb/IIIa (integrin αIIbβ3), a receptor for fibrinogen and vWF that requires inside-out activation mediated by agonist receptors, contributes to firm shear-resistant platelet adhesion and is essential for aggregate formation. The GPIb-V-IX complex is composed of 4 related transmembrane GPs: GPIbα, GPIbβ, GPV and GPIX, which are associated in a stoichiometry of 2:4:2:1 (Luo S.-Z. et al., Blood, 2007. 109(2): 603-9). Within this complex, GPIbα and GPIbβ are disulfide-linked and noncovalently associated with GPIX. GPV is noncovalently associated with GPIb-IX (Nieswandt B, et al. J Thromb Haemost. 2009; 7: 206-9). Approximately 30,000 copies of the GPIb-IX complex are found on the surface of human platelets (Varga-Szabo D, et al. J Thromb Haemost. 2009; 7: 1057-66). Loss of GPIb-V-IX function causes Bernard-Soulier syndrome (BSS), a severe bleeding disorder. BSS is characterized by abnormal, giant circulating platelets with defective adhesion to vWF and reduced thrombin responsiveness (Canobbio I, et al. Cellular signalling. 2004; 16: 1329-44). While lack or dysfunction of GPIb or GPIX are associated with BSS, no loss of function mutation in GP5 has been reported and the lack of GPV in mice does not lead to a BSS-phenotype (Ramakrishnan V, et al. PNAS. 1999; 96: 13336-41; Kahn M L, et al. Blood. 1999; 94: 4112-21). GPV is the only subunit which is not required for the correct expression of the complex (Dong J-f, et al. J Biol Chem. 1998; 273: 31449-54), GPV is highly glycosylated and contains a thrombin cleavage site leading to quantitative removal of GPV from the platelet surface and the generation of soluble GPV (sGPV) in the presence of thrombin (Ravanat C, et al. Blood. 1997; 89: 3253-62; Azorsa D O, et al. Thrombosis and Haemostasis. 1999; 81: 131-8). Of note, this thrombin cleavage site is conserved in the mouse, rat and human protein (Ravanat C, et al. Blood. 1997; 89: 3253-62). However, in contrast to protease-activated receptor (PAR) 4-deficient mice, which do not respond upon thrombin stimulation (Kahn M L, et al. Blood. 1999; 94: 4112-21; Kahn M L, et al. Nature. 1998; 394: 690-4.), Gp5^{-/-} mice display grossly normal platelet functionality.

In vitro, Gp5^{-/-} platelets are hardly distinguishable from wildtype platelets (Ramakrishnan V, et al. PNAS. 1999; 96: 13336-41; Kahn M L, et al. Blood. 1999; 94: 4112-21). Only after activation with threshold doses of thrombin, an increased responsiveness was observed (Ramakrishnan V, et al. PNAS. 1999; 96: 13336-41), which has been ascribed to the lack of GPV as an alternative substrate for thrombin competing with PARs. For one of the two Gp5^{-/-} mouse strains, reduced tail bleeding times, accelerated thrombus formation and increased embolization were reported (Ramakrishnan V, et al. PNAS. 1999; 96: 13336-41; Ni H, et al. Blood. 2001; 98: 368-73), whereas analysis of the second mouse line revealed unaltered tail bleeding times and impaired thrombus formation (Kahn M L, et al. Blood. 1999; 94: 4112-21; Moog S, et al. Blood. 2001; 98: 1038-46). The latter group ascribed the defective thrombus formation to the role of GPV in collagen signaling, thereby establishing collagen as ligand for GPV (Moog S, et al. Blood. 2001; 98: 1038-46). The latest report on Gp5^{-/-} mice used mice backcrossed to the C57Bl/6 background and confirmed the increased thrombin responsiveness as well as slightly reduced adhesion on collagen. Using laser-injury, the authors demonstrated that the effect of GPV-deficiency on thrombus formation depends on the severity of the injury and concluded that GPV is only of minor relevance for arterial thrombus formation (Nonne C, et al. J Thromb Haemost. 2008; 6: 210-2). So far, the role of GPV in thrombosis and hemostasis seems to be of minor relevance for maintaining hemostasis and platelet function. However, the exact function of GPV in thrombosis, hemostasis and thrombo-inflammatory brain infarction still remains poorly understood.

### SUMMARY OF THE INVENTION

The inventors surprisingly found that mice treated with an antibody directed against a region of the extracellular domain of GPV which is distinct from the collagen-binding site of GPV displayed an accelerated time to thrombus formation in vivo. In addition, antibody-mediated blockade of GPV could fully compensate for the lack of GPVI in in vivo thrombus formation and hemostasis.

The present invention therefore relates to the following embodiments [1] to [28]:
[1] An inhibitor of glycoprotein V (platelet glycoprotein V; GPV) for use as a coagulant.
[2] An inhibitor of glycoprotein V (platelet glycoprotein V; GPV) for use in the treatment or prevention of a hemorrhagic condition.
[3] The inhibitor for use according to embodiment [2], wherein said hemorrhagic condition is caused by a platelet disorder.
[4] The inhibitor for use according to embodiment [3], wherein said platelet disorder is characterized by a decreased number of platelets.
[5] The inhibitor for use according to embodiment [3] or [4], wherein the platelet disorder is thromobocytopenia, e.g. idiopathic thrombocytopenic purpura, thrombotic thrombocytopenic purpura, thrombocytopenia caused by chemotherapy, or immunothrombocytopenia.
[6] The inhibitor for use according to any one of embodiments [2] to [4], wherein said hemorrhagic condition is selected from the group consisting of inflammatory bleeding, hemorrhagic stroke, excessive bleeding due to sepsis, excessive bleeding due to thrombocytopenia, excessive bleeding due to disseminated intravascular coagulation (DIC), excessive bleeding due to chemotherapy, excessive bleeding due to hemolytic-uremic syndrome, excessive bleeding upon administration of soluble GPV, and excessive bleeding due to HIV infection.
[7] The inhibitor for use according to any one of the preceding embodiments, wherein said GPV is human GPV.
[8] The inhibitor for use according to embodiment [7], wherein said human GPV comprises or consists of the amino acid sequence as shown in SEQ ID NO:3.
[9] The inhibitor for use according to any one of the preceding embodiments, wherein said inhibitor is a polypeptide.
[10] The inhibitor for use according to embodiment [9], wherein said inhibitor is (i) an antibody capable of binding to the extracellular domain of GPV, (ii) a fragment or derivative of an antibody, said fragment or derivative being capable of binding to the extracellular domain of GPV, (iii) an antibody capable of binding to an epitope within the extracellular domain of GPV, or (iv) a fragment or derivative of an antibody, said fragment or derivative being capable of binding to an epitope within the extracellular domain of GPV.
[11] The inhibitor for use according to embodiment [10], wherein said extracellular domain comprises or consists of amino acids 1 to 503 of SEQ ID NO:3.
[12] The inhibitor for use according to embodiment [10] or [11], wherein said antibody, fragment or derivative binds to a region of the extracellular domain of GPV which is different from the collagen-binding site of GPV.
[13] The inhibitor for use according to any one of the embodiments [10] to [12], wherein said antibody, fragment or derivative does not delay collagen-induced aggregation.
[14] The inhibitor for use according to any one of the embodiments [10] to [13], wherein said epitope is outside the collagen-binding site of GPV and/or does not overlap with the collagen-binding site of GPV.
[15] The inhibitor for use according to any one of the embodiments [10] to [14], wherein said antibody is a monoclonal antibody or a functional fragment or functional derivative thereof.
[16] The inhibitor for use according to any one of the preceding embodiments, wherein said inhibitor is a nucleic acid.
[17] The inhibitor for use according to embodiment [16], wherein said nucleic acid is capable of reducing expression of GPV.
[18] The inhibitor for use according to embodiment [17], wherein said nucleic acid is selected from the group consisting of antisense nucleic acid, siRNA and shRNA.
[19] The inhibitor for use according to embodiment [16], wherein said nucleic acid is capable of binding to the extracellular domain of GPV.
[20] The inhibitor for use according to embodiment [19], wherein said nucleic acid is an aptamer.
[21] The inhibitor for use according to any one of the preceding embodiments, wherein said inhibitor, upon administration to a subject, does not substantially affect the number of platelets in said subject.
[22] The inhibitor for use according to any one of the embodiments [2] to [21], wherein said inhibitor is used as a coagulant.
[23] The inhibitor for use according to any one of the embodiments [2] to [22], wherein said treatment or prevention comprises administering to a subject, preferably to a human, a pharmaceutically effective amount of said inhibitor.
[24] The inhibitor for use according to embodiment [23], wherein said treatment or prevention further comprises administering to said subject a coagulant other than said inhibitor.
[25] The inhibitor for use according to embodiment [24], wherein said coagulant other than said inhibitor is selected from the group consisting of an anti-fibrinolytic agent, a platelet concentrate, a coagulation factor concentrate and fresh frozen plasma.
[26] A pharmaceutical composition comprising an inhibitor as defined in any one of the preceding embodiments, and a pharmaceutically acceptable excipient.
[27] A pharmaceutical composition as defined in embodiment [26] for use in in the treatment or prevention of a hemorrhagic condition.
[28] A method of treating a hemorrhagic condition in a subject, preferably a human, comprising administering to the subject an effective amount of an inhibitor as defined in any one of embodiments [1] to [21], or of the pharmaceutical composition of embodiment [26].

### BRIEF DESCRIPTION OF THE DRAWINGS

FIG. 1: The monoclonal anti-GPV antibody 89F12 binds to the extracellular domain of GPV. (A) 89F12-FITC binds to WT, but not to GPV-deficient platelets as assessed by flow cytometry. (B) 89F12-HRP detects recombinant murine sGPV in an ELISA system.

FIG. 2: The monoclonal anti-GPV antibody 89F12 binds to the extracellular domain of GPV without affecting platelet count and thrombin-mediated cleavage of GPV. (A, B) 100 μg 89F12 was injected intravenously and afterwards, platelet count as well as GPV surface expression were analyzed. (C) The antibody 89F12 does not affect the thrombin-mediated cleavage of GPV as assessed by flow cytometry. (D) 89F12 does not alter αIIbβ3 integrin-mediated platelet activation and P-selectin exposure in response to thrombin.

FIG. 3: 89H11, but not 89F12, blocks the collagen-binding site on GPV. Washed murine platelets from mice lacking the collagen-binding integrin α2β1 (Itga2^{-/-}) were stimulated with 2 μg/ml fibrillar collagen in the presence of vehicle (black curve) or the anti-GPV antibodies 89H11 (light grey) or 89F12 (dark grey). Displayed are representative aggregometry curves of 2 independent experiments with four mice per group each.

FIG. 4: The antibody 89F12 propagates hemostasis in wildtype mice and restores it in GPVI/CLEC-2 double-deficient animals. Displayed are tail bleeding times of the indicated mouse lines. Each symbol represents one animal. In cases where mice were unable to arrest bleeding Fisher's exact test was used to calculate P-values.

FIG. 5: The antibody 89F12 restores hemostasis in GPVI/ITGA2 double-deficient mice. Displayed are tail bleeding times of the indicated mouse lines. Each symbol represents one animal. In cases where mice were unable to arrest bleeding Fisher's exact test was used to calculate P-values.

FIG. 6: The monoclonal anti-GPV antibody 89F12 compensates for the lack of GPVI in an in vivo thrombus formation model. Mesenteric arterioles were injured with 20% FeCl₃ and adhesion and thrombus formation of fluorescently-labeled platelets were monitored by intravital microscopy. Each dot represents one vessel, horizontal lines indicate the median. * P<0.05; ** P<0.01; *** P<0.001.

FIG. 7: The antibody 89F12 can compensate for the lack of RhoA in hemostasis. Displayed are tail bleeding times of the indicated mouse lines. Each symbol represents one animal, horizontal lines indicate the median (not depicted if the median would have been above 1200 s). In cases where mice were unable to arrest bleeding Fisher's exact test was used to calculate P-values. * P<0.05; ** P<0.01.

### DETAILED DESCRIPTION

The present invention relates to an inhibitor of platelet glycoprotein V (GPV) for use as a coagulant, and/or to an inhibitor of GPV for use in the treatment or prevention of a hemorrhagic condition.

### Glycoprotein V

The term “Glycoprotein V” or “GPV”, as used herein, denotes a membrane protein having a sequence identity of at least 50% to the amino acid sequence as shown in SEQ ID NO:3. Preferably, the GPV has an amino acid identity of at least 60%, or at least 70%, or at least 80%, or at least 90%, or at least 95% to the amino acid sequence as shown in SEQ ID NO:3. The GPV has a functional transmembrane domain.

In accordance with the present invention, a sequence being evaluated (the “Compared Sequence”) has a certain “percent identity with”, or is certain “percent identical to” a claimed or described sequence (the “Reference Sequence”) after alignment of the two sequences. The “Percent Identity” is determined according to the following formula:

Percent Identity=100[1−(C/R)]

In this formula, C is the number of differences between the Reference Sequence and the Compared Sequence over the length of alignment between the two sequences wherein (i) each base in the Reference Sequence that does not have a corresponding aligned base in the Compared Sequence, and (ii) each gap in the Reference Sequence, and (iii) each aligned base in the Reference Sequence that is different from an aligned base in the Compared Sequence constitutes a difference. R is the number of bases of the Reference Sequence over the length of the alignment with the Compared Sequence with any gap created in the Reference Sequence also being counted as a base.

If an alignment exists between the Compared Sequence and the Reference Sequence for which the Percent Identity (calculated as above) is about equal to, or greater than, a specified minimum, the Compared Sequence has that specified minimum Percent Identity even if alignments may exist elsewhere in the sequence that show a lower Percent Identity than that specified.

In a preferred embodiment, the length of aligned sequence for comparison purposes is at least 30%, preferably at least 40%, more preferably at least 50%, even more preferably at least 60%, and even more preferably at least 70%, 80%, or 90% of the length of the Reference Sequence.

The comparison of sequences and determination of percent identity (and percent similarity) between two amino acid sequences can be accomplished using any suitable program, e.g. the program “BLAST 2 SEQUENCES (blastp)” (Tatusova et al. FEMS Microbiol. Lett. 1999; 174: 247-250) with the following parameters: Matrix BLOSUM62; Open gap 11 and extension gap 1 penalties; gap x_dropoff50; expect 10.0 word size 3; Filter: none. According to the present invention, the sequence comparison covers at least 40 amino acids, preferably at least 80 amino acids, more preferably at least 100 amino acids, and most preferably at least 120 amino acids.

The GPV referred to herein typically is platelet glycoprotein V.

Typically, the GPV is a naturally occurring GPV. Preferably, the GPV is of mammalian origin. Most preferably, the GPV is a human GPV. According to this embodiment, the GPV preferably comprises or consists of the amino acid sequence as shown in SEQ ID NO:3.

### Inhibitors of GPV

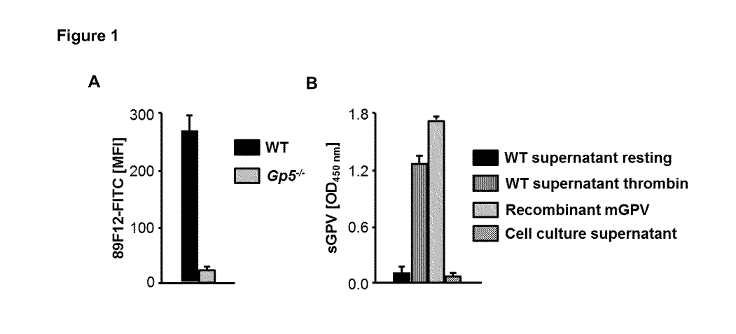

An inhibitor of GPV (hereinafter referred to as “GPV inhibitor”) is a compound which (i) has pro-coagulatory activity and (ii) is capable of binding to the extracellular domain of GPV, or of attenuating expression of GPV mRNA. Preferably, the GPV inhibitor is a compound which (i) has pro-coagulatory activity and (ii) is capable of binding to the extracellular domain of GPV. In accordance with this invention, pro-coagulatory activity is determined in a “Bleeding Time Assay” as described in the examples, with the proviso that the mouse used in the Bleeding Time Assay is a transgenic mouse lacking endogenous GPV and expressing human GPV. Binding to the extracellular domain of GPV can be determined in an ELISA as described in the examples. In one embodiment, binding to the extracellular domain of GPV is determined in an ELISA using recombinant soluble GPV, as depicted in FIG. 1B, with the proviso that recombinant soluble human GPV is used. Most preferably, binding to the extracellular domain of GPV is determined in an ELISA using recombinant soluble GPV, as depicted in FIG. 1B, with the proviso that recombinant soluble human GPV substantially consisting of amino acids 1-503 of SEQ ID NO:3 is used.

The GPV inhibitor may affect, e.g. inhibit, the thrombin-mediated cleavage of GPV in a subject upon administration of the GPV inhibitor to the subject. In another embodiment, the GPV inhibitor does not affect the thrombin-mediated cleavage of GPV in a subject upon administration of the GPV inhibitor to the subject.

The type or class of the GPV inhibitor is not particularly limited. Preferably, however, the compound is a peptide or polypeptide, more preferably the compound is an antibody or a fragment thereof. In yet another embodiment, the GPV inhibitor is a nucleic acid.

### Antibodies

In a preferred embodiment, the GPV inhibitor is an antibody. The term “antibody”, as used herein, refers to an immunoglobulin molecule that binds to or is immunologically reactive with a particular antigen, and includes polyclonal, monoclonal, genetically engineered and otherwise modified forms of antibodies including, but not limited to, chimeric antibodies, humanized antibodies, human antibodies, heteroconjugate antibodies (e.g. bispecific antibodies, diabodies, triabodies, and tetrabodies), single-domain antibodies (nanobodies) and antigen binding fragments of antibodies, including e.g. Fab′, F(ab′)2, Fab, Fv, rIgG, and scFv fragments. Moreover, unless otherwise indicated, the term “monoclonal antibody” (mAb) is meant to include both intact molecules, as well as, antibody fragments (such as, for example, Fab and F(ab′)2 fragments) which are capable of binding to a protein. Fab and F(ab′)2 fragments lack the Fc fragment of intact antibody, clear more rapidly from the circulation of the animal, and may have less non-specific tissue binding than an intact antibody (Wahl et al. J. Nucl. Med. 1983; 24: 316).

Typically, the antibody is capable of binding to the extracellular domain of GPV, preferably of human GPV. Whether a molecule or an antibody is capable of binding to the extracellular domain of GPV can be determined by a binding assay described in Example/FIG. 1A or 1B (see below).

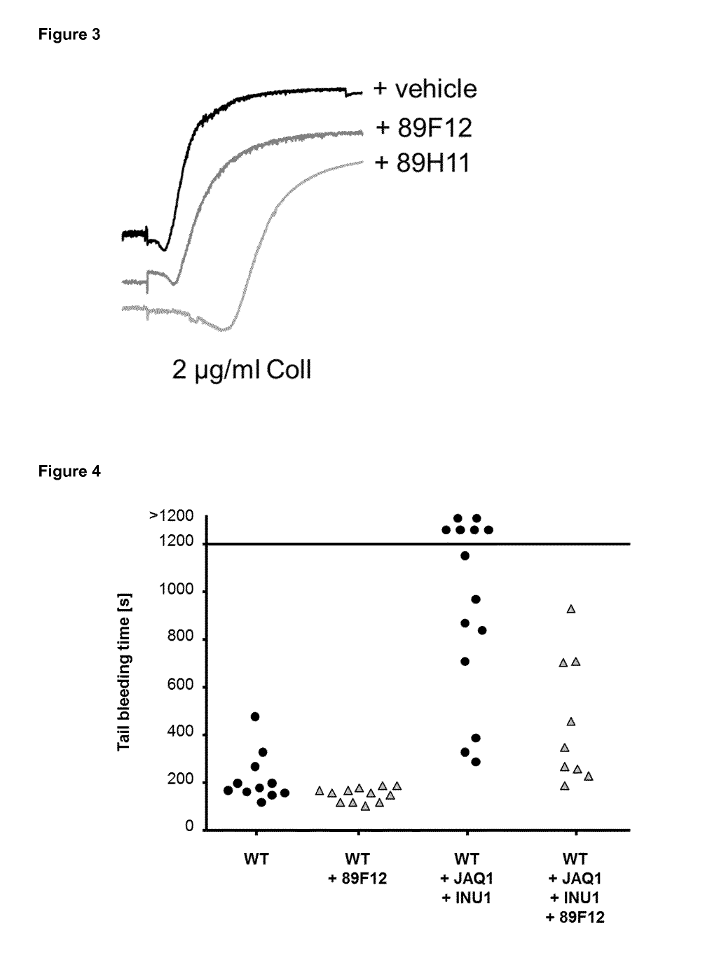

The antibody, fragment or derivative referred to herein preferably binds to a region within the extracellular domain of GPV which is distinct from the collagen-binding site of GPV. In another embodiment, the antibody, fragment or derivative does not delay collagen-induced aggregation. This can be determined in an aggregation assay as described in the Examples (see FIG. 3 and materials and methods).

Further preferred inhibitors are antibodies, fragments and derivatives thereof which compete with antibody 89F12 for binding to human GPV. Further preferred inhibitors are antibodies, fragments and derivatives thereof which bind to an epitope on GPV which overlaps with the epitope on GPV of antibody 89F12. Further preferred inhibitors are antibodies, fragments and derivatives thereof which bind to the same epitope on GPV as antibody 89F12.

In other embodiments, the inhibitor is an antibody, fragment or derivative thereof which does not compete with antibody 89H11 for binding to human GPV. In another embodiment, the inhibitor is an antibody, fragment or derivative thereof which binds to an epitope on GPV which does not overlap with the epitope on GPV of antibody 89H11. In yet another embodiment, the inhibitor is an antibody, fragment or derivative thereof which binds to an epitope on GPV which is different from the epitope on GPV of antibody 89H11.

In other embodiments, the inhibitor is an antibody, fragment or derivative thereof which does not compete with antibody V.3 (Azorsa D O, et al. Thrombosis and Haemostasis. 1999; 81: 131-8; Moog S, et al. Blood. 2001; 98: 1038-46) for binding to human GPV. In another embodiment, the inhibitor is an antibody, fragment or derivative thereof which binds to an epitope on GPV which does not overlap with the epitope on GPV of antibody V.3. In yet another embodiment, the inhibitor is an antibody, fragment or derivative thereof which binds to an epitope on GPV which is different from the epitope on GPV of antibody V.3.

The embodiments in the preceding paragraphs can be combined with each other.

The antibody preferably binds to an epitope within amino acids 1-503 of SEQ ID NO:3. For example, the present invention includes, but is not limited to, the following embodiments:

TABLE 1

| | The antibody binds to an epitope within the |
|---|---|
| Embodiment No. | following amino acids of SEQ ID NO: 3 |
| 1 | 1-30 |
| 2 | 16-45 |
| 3 | 31-60 |
| 4 | 46-75 |
| 5 | 61-90 |
| 6 | 76-105 |
| 7 | 91-120 |
| 8 | 106-135 |
| 9 | 121-150 |
| 10 | 136-165 |
| 11 | 151-180 |
| 12 | 166-195 |
| 13 | 181-210 |
| 14 | 196-225 |
| 15 | 211-240 |
| 16 | 226-255 |
| 17 | 241-270 |
| 18 | 256-285 |
| 19 | 271-300 |
| 20 | 286-315 |
| 21 | 301-330 |
| 22 | 316-345 |
| 23 | 331-360 |
| 24 | 346-375 |
| 25 | 361-390 |
| 26 | 376-405 |
| 27 | 391-420 |
| 28 | 406-435 |
| 29 | 421-450 |
| 30 | 436-465 |
| 31 | 451-480 |
| 32 | 466-495 |
| 33 | 481-503 |

The dissociation constant K_{D} for the complex formed by the extracellular domain of GPV and antibody is preferably less than 100 μM, more preferably less than 10 μM, most preferably less than 5 μM. Typically the K_{D} ranges from about 1 μM to about 10 μM, or from about 10 μM to about 1 μM, or from about 100 μM to about 100 nM. Preferably, the antibody-GPV complex has a K_{D} in the range from 5 μM to 1 nM, most preferably from 10 pM to 500 pM.

Preferably, the antibody is a monoclonal antibody. The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. Monoclonal antibodies can be prepared using a wide variety of techniques known in the art including the use of hybridoma, recombinant, and phage display technologies, or a combination thereof (Harlow and Lane, “Antibodies, A Laboratory Manual” CSH Press 1988, Cold Spring Harbor N.Y.).

In other embodiments, including in vivo use of the anti-GPV antibodies in humans, chimeric, primatized, humanized, or human antibodies can be used. In a preferred embodiment, the antibody is a human antibody or a humanized antibody, more preferably a monoclonal human antibody or a monoclonal humanized antibody.

The term “chimeric” antibody as used herein refers to an antibody having variable sequences derived from non-human immunoglobulins, such as rat or mouse antibodies, and human immunoglobulins constant regions, typically chosen from a human immunoglobulin template. Methods for producing chimeric antibodies are known in the art. See, e.g. Morrison. Science. 1985; 229(4719): 1202-7; Oi et al. Bio Techniques. 1986; 4: 214-221; Gillies et al. J. Immunol. Methods 1985; 125: 191-202; U.S. Pat. Nos. 5,807,715; 4,816,567; and 4,816,397, which are incorporated herein by reference in their entireties.

“Humanized” forms of non-human (e.g. murine) antibodies are chimeric immunoglobulins, immunoglobulin chains or fragments thereof (such as Fv, Fab, Fab′, F(ab′)2 or other target-binding subsequences of antibodies), which contain minimal sequences derived from a non-human immunoglobulin. In general, the humanized antibody will comprise substantially all of at least one and typically two variable domains, in which all or substantially all of the complementarity determining regions (CDRs) correspond to those of a non-human immunoglobulin and all or substantially all of the framework (FR) regions are those of a human immunoglobulin consensus sequence. The humanized antibody can also comprise at least a portion of an immunoglobulin constant region (Fc), typically that of a human immunoglobulin template chosen. Humanization is a technique for making a chimeric antibody in which one or more amino acids or portions of the human variable domain have been substituted by the corresponding sequence from a non-human species. Humanized antibodies are antibody molecules generated in a non-human species that bind the desired antigen having one or more CDRs from the non-human species and FRs from a human immunoglobulin molecule. Often, framework residues in the human framework regions will be substituted with the corresponding residue from the CDR donor antibody to alter, preferably improve, antigen binding. These framework substitutions are identified by methods well known in the art, e.g. by modeling of the interactions of the CDR and framework residues to identify framework residues important for antigen binding and sequence comparison to identify unusual framework residues at particular positions. See, e.g. Riechmann et al. Nature 1988. 332: 323-7 and Queen et al. U.S. Pat. Nos. 5,530,101; 5,585,089; 5,693,761; 5,693,762; and 6,180,370 (each of which is incorporated by reference in its entirety). Antibodies can be humanized using a variety of techniques known in the art including, for example, CDR-grafting (EP239400; WO 91/09967; U.S. Pat. Nos. 5,225,539; 5,530,101 and 5,585,089), veneering or resurfacing (EP0592106; EP0519596; Padlan. Mol Immunol. 1991; 28: 489-498; Studnicka et al. Prot Eng. 1994; 7: 805-814; Roguska et al. PNAS 1994; 91: 969-973, and chain shuffling (U.S. Pat. No. 5,565,332)), all of which are hereby incorporated by reference in their entireties.

In some embodiments, humanized antibodies are prepared as described in Queen et al., U.S. Pat. Nos. 5,530,101; 5,585,089; 5,693,761; 5,693,762; and 6,180,370 (each of which is incorporated by reference in its entirety).

In some embodiments, the anti-GPV antibodies are human antibodies. Completely “human” anti-GPV antibodies can be desirable for therapeutic treatment of human patients. As used herein, “human antibodies” include antibodies having the amino acid sequence of a human immunoglobulin and include antibodies isolated from human immunoglobulin libraries or from animals transgenic for one or more human immunoglobulin and that do not express endogenous immunoglobulins. Human antibodies can be made by a variety of methods known in the art including phage display methods described above using antibody libraries derived from human immunoglobulin sequences. See U.S. Pat. Nos. 4,444,887 and 4,716,111; and WO 98/46645; WO 98/50433; WO 98/24893; WO 98/16654; WO 96/34096; WO 96/33735; and WO 91/10741, each of which is incorporated herein by reference in its entirety. Human antibodies can also be produced using transgenic mice which are incapable of expressing functional endogenous immunoglobulins, but which can express human immunoglobulin genes. See, e.g. WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entireties. Completely human antibodies that recognize a selected epitope can be generated using a technique referred to as “guided selection.” In this approach a selected non-human monoclonal antibody, e.g. a mouse antibody, is used to guide the selection of a completely human antibody recognizing the same epitope (Jespers et al. Biotechnology. 1988; 12: 899-903).

In some embodiments, the anti-GPV antibodies are primatized antibodies. The term “primatized antibody” refers to an antibody comprising monkey variable regions and human constant regions. Methods for producing primatized antibodies are known in the art. See e.g. U.S. Pat. Nos. 5,658,570; 5,681,722; and 5,693,780, which are incorporated herein by reference in their entireties.

In some embodiments, the anti-GPV antibodies are derivatized antibodies. For example, but not by way of limitation, the derivatized antibodies that have been modified, e.g. by glycosylation, acetylation, pegylation, phosphorylation, amidation, derivatization by known protecting/blocking groups, proteolytic cleavage or linkage to a cellular ligand or other proteins (see below for a discussion of antibody conjugates). Any of numerous chemical modifications may be carried out by known techniques, including, but not limited to, specific chemical cleavage, acetylation, formylation or metabolic synthesis of tunicamycin, etc. Additionally, the derivative may contain one or more non-classical amino acids.

In some embodiments, the anti-GPV antibodies or fragments thereof can be antibodies or antibody fragments, whose sequence has been modified to reduce at least one constant region-mediated biological effector function relative to the corresponding wild type sequence. To modify an anti-GPV antibody, such that it exhibits reduced binding to the Fc receptor, the immunoglobulin constant region segment of the antibody can be mutated at particular regions necessary for Fc receptor (FcR) interactions (see, e.g. Canfield and Morrison. J Exp Med. 1991; 173: 1483-1491; and Lund et al. J Immunol. 1991; 147: 2657-2662). Reduction in FcR binding ability of the antibody can also reduce other effector functions which rely on FcR interactions, such as opsonization, phagocytosis and antigen-dependent cellular cytotoxicity.

In yet another aspect, the anti-GPV antibodies or fragments thereof can be antibodies or antibody fragments that have been modified to increase or reduce their binding affinities to the fetal Fc receptor, FcRn. To alter the binding affinity to FcRn, the immunoglobulin constant region segment of the antibody can be mutated at particular regions necessary for FcRn interactions (see, e.g. WO 2005/123780). Increasing the binding affinity to FcRn should increase the antibody's serum half-life, and reducing the binding affinity to FcRn should conversely reduce the antibody's serum half-life. Specific combinations of suitable amino acid substitutions are identified in Table 1 of WO 2005/123780, which table is incorporated by reference herein in its entirety. See also, Hinton et al., U.S. Pat. Nos. 7,217,797, 7,361,740, 7,365,168, and 7,217,798, which are incorporated herein by reference in their entireties. In yet other aspects, an anti-GPV antibody has one or more amino acids inserted into one or more of its hypervariable regions, for example as described in US 2007/0280931.

### Antibody Conjugates

In some embodiments, the anti-GPV antibodies are antibody conjugates that are modified, e.g. by the covalent attachment of any type of molecule to the antibody, such that covalent attachment does not interfere with binding to GPV, Techniques for conjugating effector moieties to antibodies are well known in the art (See, e.g. Hellstrom et al., Controlled Drag Delivery, 2nd Ed., 623-53 (Robinson et al., eds., 1987); Thorpe et al, Immunol Rev. 1982; 62: 119-58 and Dubowchik et al. Pharmacology and Therapeutics 1999; 83: 67-123).

In one example, the antibody or fragment thereof is fused via a covalent bond (e.g. a peptide bond), at optionally the N-terminus or the C-terminus, to an amino acid sequence of another protein (or portion thereof; preferably at least a 10, 20 or 50 amino acid portion of the protein). Preferably, the antibody or fragment thereof is linked to the other protein at the N-terminus of the constant domain of the antibody. Recombinant DNA procedures can be used to create such fusions, for example as described in WO 86/01533 and EP 0392745. In another example, the effector molecule can increase half-life in vivo. Examples of suitable effector molecules of this type include polymers, albumin, albumin binding proteins or albumin binding compounds, such as those described in WO 2005/117984.

In some embodiments, anti-GPV antibodies can be attached to poly(ethyleneglycol) (PEG) moieties. For example, if the antibody is an antibody fragment, the PEG moieties can be attached through any available amino acid side-chain or terminal amino acid functional group located in the antibody fragment, for example any free amino, imino, thiol, hydroxyl or carboxyl group. Such amino acids can occur naturally in the antibody fragment or can be engineered into the fragment using recombinant DNA methods. See, for example U.S. Pat. No. 5,219,996. Multiple sites can be used to attach two or more PEG molecules. Preferably, PEG moieties are covalently linked through a thiol group of at least one cysteine residue located in the antibody fragment. Where a thiol group is used as the point of attachment, appropriately activated effector moieties, for example thiol selective derivatives, such as maleimides and cysteine derivatives, can be used.

In another example, an anti-GPV antibody conjugate is a modified Fab′ fragment which is PEGylated, i.e., has PEG (poly(ethyleneglycol)) covalently attached thereto, e.g. according to the method disclosed in EP 0948544. See also Poly(ethyleneglycol) Chemistry, Biotechnical and Biomedical Applications, (J. Milton Harris (ed.), Plenum Press, New York, 1992); Poly(ethyleneglycol) Chemistry and Biological Applications, (J. Milton Harris and S. Zalipsky, eds., American Chemical Society, Washington D. C, 1997); and Bioconjugation Protein Coupling Techniques for the Biomedical Sciences, (M. Aslam and A. Dent, eds., Grove Publishers, New York, 1998); and Chapman. Advanced Drug Delivery Reviews 2002; 54: 531-545.

### Nucleic Acids

In other embodiments of the invention, the GPV inhibitor is a nucleic acid. In a particular embodiment, the nucleic acid is capable of attenuating expression of GPV mRNA. Expression of GPV can be inhibited or reduced or abolished by RNA interference, e.g. by using siRNA or shRNA. Another possibility is the use of antisense nucleic acid, e.g. antisense RNA.

The phrase “attenuating expression of an mRNA,” as used herein, means administering or expressing an amount of interfering RNA (e.g. an siRNA) to reduce translation of the target mRNA into protein, either through mRNA cleavage or through direct inhibition of translation. The reduction in expression of the target mRNA or the corresponding protein is commonly referred to as “knock-down” and is reported relative to levels present following administration or expression of a non-targeting control RNA (e.g. a non-targeting control siRNA). Knock-down of expression of an amount including and between 50% and 100% is contemplated by embodiments herein. However, it is not necessary that such knock-down levels are achieved for purposes of the present invention. Knock-down is commonly assessed by measuring the mRNA levels using quantitative polymerase chain reaction (qPCR) amplification or by measuring protein levels by western blot or enzyme-linked immunosorbent assay (ELISA). Analyzing the protein level provides an assessment of both mRNA cleavage as well as translation inhibition. Further techniques for measuring knock-down include RNA solution hybridization, nuclease protection, northern hybridization, gene expression monitoring with a microarray, antibody binding, radioimmunoassay, and fluorescence activated cell analysis.

Inhibition of GPV mRNA expression may also be determined in vitro by evaluating GPV mRNA levels or GPV protein levels in, for example, human cells following transfection of GPV-interfering RNA.

In one embodiment of the invention, interfering RNA (e.g. siRNA) has a sense strand and an antisense strand, and the sense and antisense strands comprise a region of at least near-perfect contiguous complementarity of at least 19 nucleotides. In a further embodiment of the invention, interfering RNA (e.g. siRNA) has a sense strand and an antisense strand, and the antisense strand comprises a region of at least near-perfect contiguous complementarity of at least 19 nucleotides to a target sequence of GPV mRNA, and the sense strand comprises a region of at least near-perfect contiguous identity of at least 19 nucleotides with a target sequence of GPV mRNA, respectively. In a further embodiment of the invention, the interfering RNA comprises a region of at least 13, 14, 15, 16, 17, or 18 contiguous nucleotides having percentages of sequence complementarity to or, having percentages of sequence identity with, the penultimate 13, 14, 15, 16, 17, or 18 nucleotides, respectively, of the 3′ end of the corresponding target sequence within an mRNA.

The length of each strand of the interfering RNA comprises 19 to 49 nucleotides, and may comprise a length of 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 nucleotides. The antisense strand of an siRNA is the active guiding agent of the siRNA in that the antisense strand is incorporated into RISC, thus allowing RISC to identify target mRNAs with at least partial complementary to the antisense siRNA strand for cleavage or translational repression. In embodiments of the present invention, interfering RNA target sequences (e.g. siRNA target sequences) within the GPV mRNA sequence are selected using available design tools. Interfering RNAs corresponding to a GPV target sequence are then tested by transfection of cells expressing the target mRNA followed by assessment of knockdown as described above. Techniques for selecting target sequences for siRNAs are provided in “siRNA Design: Methods and Protocols”, edited by Debra J. Taxman, 2012 (ISBN-13: 9781627031189).

In a second particular embodiment, the nucleic acid is capable of binding to the extracellular domain of human GPV. In accordance with this embodiment, the nucleic acid is preferably an aptamer. Aptamers can be designed as described in “De novo Molecular Design”, 2013, edited by Gisbert Schneider, Chapter 21 (ISBN-13: 9783527677030); or in “The Aptamer Handbook: Functional Oligonucleotides and Their Applications”, 2006, edited by Sven Klussmann (ISBN-13: 9783527607914).

### Hemorrhagic Conditions

The inhibitor described herein is preferably used in the treatment or prevention of a hemorrhagic condition. Hemorrhagic conditions are characterized by excessive bleeding. The excessive bleeding can have various causes.

In one embodiment, the hemorrhagic condition is caused by a platelet disorder.

The platelet disorder may be characterized by a decreased number of platelets, e.g. in the case of thrombocytopenia. Specific thrombocytopenias include, but are not limited to, idiopathic thrombocytopenic purpura, thrombotic thrombocytopenic purpura, drug-induced thrombocytopenia due to immune-mediated platelet destruction (e.g. by heparin, trimethoprim/sulfamethoxazole), drug-induced thrombocytopenia due to dose-dependent bone marrow suppression (e.g. by chemotherapeutic agents), thrombocytopenia accompanying systemic infection, thrombocytopenia caused by chemotherapy, gestational thrombocytopenia, and immune thrombocytopenia (ITP, formerly called immune thrombocytopenic purpura).

The platelet disorder may be characterized by a dysfunction of the platelets, e.g. in the case of defective platelet signaling due to lack of platelet receptors or signaling molecules.

In a preferred embodiment, the hemorrhagic condition is selected from the group consisting of inflammatory bleeding, hemorrhagic stroke, excessive bleeding due to sepsis, excessive bleeding due to thrombocytopenia, excessive bleeding due to disseminated intravascular coagulation (DIC), excessive bleeding due to chemotherapy, excessive bleeding due to hemolytic-uremic syndrome, and excessive bleeding due to HIV infection.

In a specific embodiment, the present invention relates to the use of the inhibitor described herein as antidote for the administration of soluble GPV.

### Pharmaceutical Compositions and Treatment

Treatment of a disease encompasses the treatment of patients already diagnosed as having any form of the disease at any clinical stage or manifestation; the delay of the onset or evolution or aggravation or deterioration of the symptoms or signs of the disease; and/or preventing and/or reducing the severity of the disease.

A “subject” or “patient” to whom a GPV inhibitor, e.g. an anti-GPV antibody, is administered can be a mammal, such as a non-primate (e.g. cow, pig, horse, cat, dog, rat, etc.) or a primate (e.g. monkey or human). In certain aspects, the human is a pediatric patient. In other aspects, the human is an adult patient.

Compositions comprising a GPV inhibitor and, optionally one or more additional therapeutic agents, such as the second therapeutic agents described below, are described herein. The compositions typically are supplied as part of a sterile, pharmaceutical composition that includes a pharmaceutically acceptable carrier. This composition can be in any suitable form (depending upon the desired method of administering it to a patient).

The GPV inhibitors, e.g. the anti-GPV antibodies, can be administered to a patient by a variety of routes such as orally, transdermally, subcutaneously, intranasally, intravenously, intramuscularly, intrathecally, topically or locally. The most suitable route for administration in any given case will depend on the particular antibody, the subject, and the nature and severity of the disease and the physical condition of the subject. Typically, a GPV inhibitor, e.g. an anti-GPV antibody, will be administered intravenously.

Another aspect of the invention is a pharmaceutical composition comprising the antibody or antigen-binding fragment thereof of the invention. The antibody or antigen-binding fragment thereof can be formulated according to known methods for preparing a pharmaceutical composition. For example, it can be mixed with one or more pharmaceutically acceptable carriers, diluents or excipients. For example, sterile water or physiological saline may be used. Other substances, such as pH buffering solutions, viscosity reducing agents, or stabilizers may also be included.

A wide variety of pharmaceutically acceptable excipients and carriers are known in the art. Such pharmaceutical carriers and excipients as well as suitable pharmaceutical formulations have been amply described in a variety of publications (see for example “Pharmaceutical Formulation Development of Peptides and Proteins”, Frokjaer et al., Taylor & Francis (2000) or “Handbook of Pharmaceutical Excipients”, 3rd edition, Kibbe et al., Pharmaceutical Press (2000) A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7th ed., Lippincott, Williams, & Wilkins; and Handbook of Pharmaceutical Excipients (2000) A, H. Kibbe et al., eds., 3rd ed. Amer. Pharmaceutical Assoc). In particular, the pharmaceutical composition comprising the antibody of the invention may be formulated in lyophilized or stable soluble form. The polypeptide may be lyophilized by a variety of procedures known in the art. Lyophilized formulations are reconstituted prior to use by the addition of one or more pharmaceutically acceptable diluents such as sterile water for injection or sterile physiological saline solution.

The pharmaceutical composition of the invention can be administered in dosages and by techniques well known in the art. The amount and timing of the administration will be determined by the treating physician or veterinarian to achieve the desired purposes. The route of administration can be via any route that delivers a safe and therapeutically effective dose to the blood of the subject to be treated. Possible routes of administration include systemic, topical, enteral and parenteral routes, such as intravenous, intraarterial, subcutaneous, intradermal, intraperitoneal, oral, transmucosal, epidural, or intrathecal. Preferred routes are intravenous or subcutaneous.

The effective dosage and route of administration are determined by factors, such as age and weight of the subject, and by the nature and therapeutic range of the antibody or antigen-binding fragment thereof. The determination of the dosage is determined by known methods, no undue experimentation is required.

A therapeutically effective dose is a dose of the antibody or antigen binding fragment thereof of the invention that brings about a positive therapeutic effect in the patient or subject requiring the treatment. A therapeutically effective dose is in the range of about 0.01 to 50 mg/kg, from about 0.01 to 30 mg/kg, from about 0.1 to 30 mg/kg, from about 0.1 to 10 mg/kg, from about 0.1 to 5 mg/kg, from about 1 to 5 mg/kg, from about 0.1 to 2 mg/kg or from about 0.1 to 1 mg/kg. The treatment may comprise giving a single (e.g. bolus) dose or multiple doses. Alternatively continuous administration is possible. If multiple doses are required, they may be administered daily, every other day, weekly, biweekly, monthly, or bimonthly or as required. A depository may also be used that slowly and continuously releases the antibody or antigen-binding fragment thereof. A therapeutically effective dose may be a dose that inhibits GPV in the subject by at least 50%, preferably by at least 60%, 70%, 80%, 90%, more preferably by at least 95%, 99% or even 100%.

The antibody can be formulated as an aqueous solution.

Pharmaceutical compositions can be conveniently presented in unit dose forms containing a predetermined amount of a GPV inhibitor, e.g. an anti-GPV antibody, per dose. Such a unit can contain 0.5 mg to 5 g, for example, but without limitation, 1 mg, 10 mg, 20 mg, 30 mg, 40 mg, 50 mg, 100 mg, 200 mg, 300 mg, 400 mg, 500 mg, 750 mg, 1000 mg, or any range between any two of the foregoing values, for example 10 mg to 1000 mg, 20 mg to 50 mg, or 30 mg to 300 mg. Pharmaceutically acceptable carriers can take a wide variety of forms depending, e.g. on the condition to be treated or route of administration.

Determination of the effective dosage, total number of doses and length of treatment with a GPV inhibitor, e.g. an anti-GPV antibody, is well within the capabilities of those skilled in the art and can be determined using a standard dose escalation study.

Therapeutic formulations of the GPV inhibitors, e.g. the anti-GPV antibodies, suitable in the methods described herein can be prepared for storage as lyophilized formulations or aqueous solutions by mixing the inhibitor, e.g. the antibody, having the desired degree of purity with optional pharmaceutically-acceptable carriers, excipients or stabilizers typically employed in the art (all of which are referred to herein as “carriers”), i.e. buffering agents, stabilizing agents, preservatives, isotonifiers, non-ionic detergents, antioxidants and other miscellaneous additives. See, Remington's Pharmaceutical Sciences, 16th edition (Osol, ed. 1980). Such additives must be nontoxic to the recipients at the dosages and concentrations employed.

Buffering agents help to maintain the pH in the range which approximates physiological conditions. They can be present at concentrations ranging from about 2 mM to about 50 mM. Suitable buffering agents include both organic and inorganic acids and salts thereof, such as citrate buffers (e.g. monosodium citrate-disodium citrate mixture, citric acid-trisodium citrate mixture, citric acid-monosodium citrate mixture, etc.), succinate buffers (e.g. succinic acid-monosodium succinate mixture, succinic acid-sodium hydroxide mixture, succinic acid-disodium succinate mixture, etc.), tartrate buffers (e.g. tartaric acid-sodium tartrate mixture, tartaric acid-potassium tartrate mixture, tartaric acid-sodium hydroxide mixture, etc.), fumarate buffers (e.g. fumaric acid-monosodium fumarate mixture, fumaric acid-disodium fumarate mixture, monosodium fumarate-disodium fumarate mixture, etc.), gluconate buffers (e.g. gluconic acid-sodium gluconate mixture, gluconic acid-sodium hydroxide mixture, gluconic acid-potassium gluconate mixture, etc.), oxalate buffer (e.g. oxalic acid-sodium oxalate mixture, oxalic acid-sodium hydroxide mixture, oxalic acid-potassium oxalate mixture, etc), lactate buffers (e.g. lactic acid-sodium lactate mixture, lactic acid-sodium hydroxide mixture, lactic acid-potassium lactate mixture, etc.) and acetate buffers (e.g. acetic acid-sodium acetate mixture, acetic acid-sodium hydroxide mixture). Additionally, phosphate buffers, histidine buffers and trimethylamine salts, such as Tris can be used.

Preservatives can be added to retard microbial growth, and can be added in amounts ranging from 0.2%-1% (w/v). Suitable preservatives include phenol, benzyl alcohol, meta-cresol, methyl paraben, propyl paraben, octadecyldimethylbenzyl ammonium chloride, benzalconium halides (e.g. chloride, bromide, and iodide), hexamethonium chloride, and alkyl parabens, such as methyl or propyl paraben, catechol, resorcinol, cyclohexanol, and 3-pentanol. Isotonicifiers sometimes known as “stabilizers” can be added to ensure isotonicity of liquid compositions and include polhydric sugar alcohols, preferably trihydric or higher sugar alcohols, such as glycerin, erythritol, arabitol, xylitol, sorbitol and mannitol. Stabilizers refer to a broad category of excipients, which can range in function from a bulking agent to an additive, which solubilizes the therapeutic agent or helps to prevent denaturation or adherence to the container wall. Typical stabilizers can be polyhydric sugar alcohols (enumerated above); amino acids, such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols, such as inositol; polyethylene glycol; amino acid polymers; sulfur containing reducing agents, such as urea, glutathione, thioctic acid, sodium thioglycolate, thioglycerol, α-monothioglycerol and sodium thio sulfate; low molecular weight polypeptides (e.g. peptides of 10 residues or fewer); proteins, such as human serum albumin, bovine serum albumin, gelatin or immunoglobulins; hydrophylic polymers, such as polyvinylpyrrolidone monosaccharides, such as xylose, mannose, fructose, glucose; disaccharides, such as lactose, maltose, sucrose and trisaccacharides, such as raffinose; and polysaccharides, such as dextran. Stabilizers can be present in the range from 0.1 to 10,000 weights per part of weight active protein.

Non-ionic surfactants or detergents (also known as “wetting agents”) can be added to help solubilize the therapeutic agent as well as to protect the therapeutic protein against agitation-induced aggregation, which also permits the formulation to be exposed to shear surface stressed without causing denaturation of the protein. Suitable non-ionic surfactants include polysorbates (20, 80, etc.), polyoxamers (184, 188, etc.), pluronic polyols, polyoxyethylene sorbitan monoethers (TWEEN®-20, TWEEN®-80, etc.). Non-ionic surfactants can be present in a range of about 0.05 mg/ml to about 1.0 mg/ml, or in a range of about 0.07 mg/ml to about 0.2 mg/ml.

Additional miscellaneous excipients include bulking agents (e.g. starch), chelating agents (e.g. EDTA), antioxidants (e.g. ascorbic acid, methionine, vitamin E), and co-solvents.

The formulation herein can also contain a second therapeutic agent in addition to a GPV inhibitor, e.g. an anti-GPV antibody. Examples of suitable second therapeutic agents are provided below.

The dosing schedule can vary from once a month to daily depending on a number of clinical factors, including the type of disease, severity of disease, and the patient's sensitivity to the GPV inhibitor, e.g. an anti-GPV antibody. In specific embodiments, a GPV inhibitor, e.g. an anti-GPV antibody, is administered daily, twice weekly, three times a week, every 5 days, every 10 days, every two weeks, every three weeks, every four weeks or once a month, or in any range between any two of the foregoing values, for example from every four days to every month, from every 10 days to every two weeks, or from two to three times a week, etc. The dosage of a GPV inhibitor, e.g. an anti-GPV antibody, to be administered will vary according to the particular antibody, the subject, and the nature and severity of the disease, the physical condition of the subject, the therapeutic regimen (e.g. whether a second therapeutic agent is used), and the selected route of administration; the appropriate dosage can be readily determined by a person skilled in the art.

It will be recognized by one of skill in the art that the optimal quantity and spacing of individual dosages of a GPV inhibitor, e.g. an anti-GPV antibody, will be determined by the nature and extent of the condition being treated, the form, route and site of administration, and the age and condition of the particular subject being treated, and that a physician will ultimately determine appropriate dosages to be used. This dosage can be repeated as often as appropriate. If side effects develop, the amount and/or frequency of the dosage can be altered or reduced, in accordance with normal clinical practice.

### Combination Therapy

Preferably, the patient being treated with the GPV inhibitor, e.g. an anti-GPV antibody, is also treated with conventional coagulants. For example, a patient suffering from excessive bleeding is typically also being treated with an anti-fibrinolytic agent, a platelet concentrate, a coagulation factor concentrate and/or fresh frozen plasma.

Yet another aspect of the invention is the use of an inhibitor (preferably an antibody) as defined hereinabove for promoting hemostasis.

Yet another aspect of the invention is a compound (preferably an antibody) as defined hereinabove for use in reducing the bleeding time in a patient suffering from excessive bleeding.

The invention further relates to a method of reducing the bleeding time, comprising administering to a subject an effective amount of an inhibitor (preferably an antibody) as defined hereinabove.

A further aspect of this invention is a method of treating a hemorrhagic condition, comprising administering to a patient in need thereof an effective amount of an inhibitor (preferably an antibody) as defined hereinabove. The hemorrhagic condition is preferably one of the conditions described above.

A further aspect of this invention is a method of preventing a hemorrhagic condition, comprising administering to a patient in need thereof an effective amount of an inhibitor (preferably an antibody) as defined hereinabove. The hemorrhagic condition is preferably one of the conditions described above.

Overview of the nucleotide and amino acid sequences:

| SEQ ID NO: | Description |
|---|---|
| 1 | Nucleic acid sequence encoding human GPV |
| 2 | Amino acid sequence encoded by SEQ ID NO: 1, |
| | i.e. human GPV including signal peptide |
| 3 | Amino acid sequence of human GPV lacking signal peptide |
| 4 | Nucleic acid sequence encoding murine GPV |
| 5 | Amino acid sequence encoded by SEQ ID NO: 4, |
| | i.e. murine GPV including signal peptide |
| 6 | Amino acid sequence of murine GPV lacking signal peptide |

### EXAMPLES

### Results

89F12 is a monoclonal rat anti-mouse GPV antibody. It was generated by fusion of immortalized AG14 myeloma cells and spleen cells of rats, which were immunized with mouse platelets. 89F12 binds to wildtype, but not to GPV-deficient mouse platelets (FIG. 1A) and 89F12 binds soluble GPV which is formed upon thrombin-cleavage of the receptor (FIG. 1B). Moreover, 89F12 binds to the recombinantly expressed extracellular domain of murine GPV (FIG. 1B).

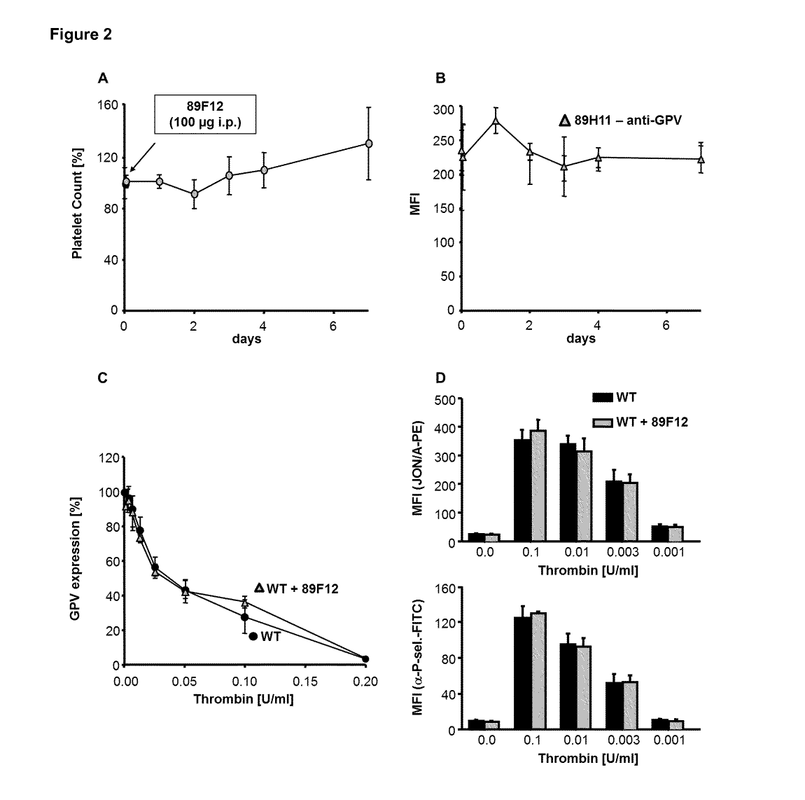

In vivo, 89F12-IgG or 89F12-Fab fragments have no effect on the platelet count and have no influence on the thrombin-mediated cleavage of GPV (FIG. 2 and data not shown). Furthermore, 89F12 does not affect platelet activation in vitro as assessed by flow cytometry (FIG. 2D), aggregometry or flow adhesion assays (not shown). Of note it does not interfere with the collagen-binding site of GPV as it does not delay collagen-induced aggregation (FIG. 3)—in contrast to 89H11, an anti-GPV antibody which blocks the collagen-binding site of GPV.

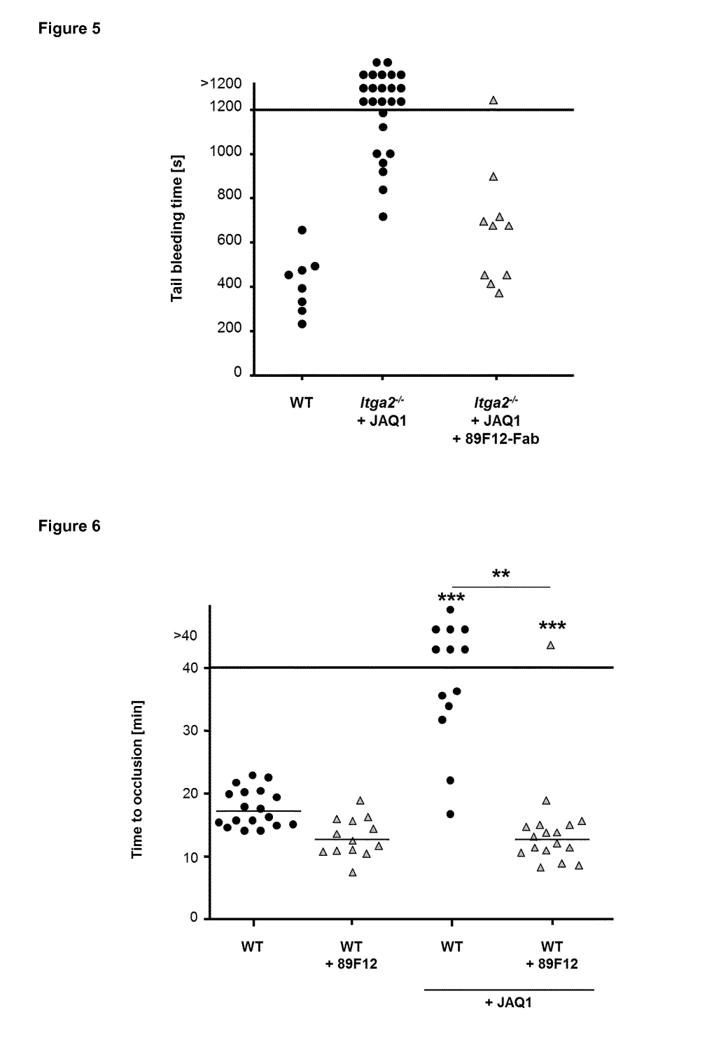

The effect of the anti-GPV antibody on hemostasis and thrombus formation was tested in vivo. Intravenous injection of 100 μg 89F12 in WT mice resulted in an accelerated time to cessation of the blood loss in the tail bleeding time assay. Simultaneous depletion of GPVI and CLEC-2 by JAQ1- and INU1-injection, respectively, resulted in a pronounced hemostatic defect since 5 of 14 mice could not stop the bleeding within the observation period of 20 min. Blockade of the extracellular domain of GPV by 89F12 could compensate for the hemostatic defect of GPVI/CLEC-2 double-depleted mice resulting in tail bleeding times in these mice comparable to 89F12-treated WT mice. This means that 89F12 can restore hemostasis in GPVI/CLEC-2 double-depleted mice. Thereby, 89F12 prevents hemostatic complications in the absence of GPVI and CLEC-2 (FIG. 4). To test whether receptor dimerization or the Fc portion of the antibody are required for its effects on thrombus formation GPVI-depleted α2-deficient (Itga2^{-/-}) mice were treated with vehicle or 89F12-Fab fragments. Vehicle-treated GPVI-depleted Itga2^{-/-} mice bled more than 20 min, while 89F12-Fab fragments restored the hemostatic function of these mice (FIG. 5).

Since GPV blockade promotes hemostasis, thrombus formation was also studied in 89F12-treated mice. 89F12-treated mice displayed an accelerated time to thrombus formation in vivo. Depletion of the principal platelet activating collagen receptor GPVI using JAQ1 led to a delayed thrombus formation time in WT mice and only 6 out of 13 vessels occluded within the observation period. Even in the absence of GPVI, 89F12-mediated blockade of GPV resulted in an earlier beginning of thrombus formation (data not shown) finally leading to a faster vessel occlusion even if compared to untreated WT controls (P<0.001, FIG. 6). 89F12-mediated blockade of GPV could fully compensate for the lack of GPVI in in vivo thrombus formation and hemostasis.

The fact that GPV-blockade could counterbalance the absence of the (hem)ITAM receptors GPVI and CLEC-2 suggests that GPV serves as a general negative regulator of platelet activation. Therefore, we investigated whether GPV blockade could compensate for the absence of other critical signaling molecules.

Platelet activation upon vessel wall injury leads to cytoskeletal rearrangements which are crucial for conversion from a discoid to a spheric platelet shape, for granule secretion and spreading. The Rho family of small GTPases, such as RhoA, is thought to be involved in many of these processes. RhoA plays a central role in the organization of the actin cytoskeleton through the formation of stress fibers, the regulation of actomyosin contractility and in the regulation of microtubule dynamics (Bustelo X R, et al. BioEssays, 2007; 29: 356-70). Furthermore, RhoA is involved in different cellular processes downstream of G_{q}- and G₁₃-coupled agonist receptors (Pleines I, et al. Blood. 2012; 119: 1054-63).

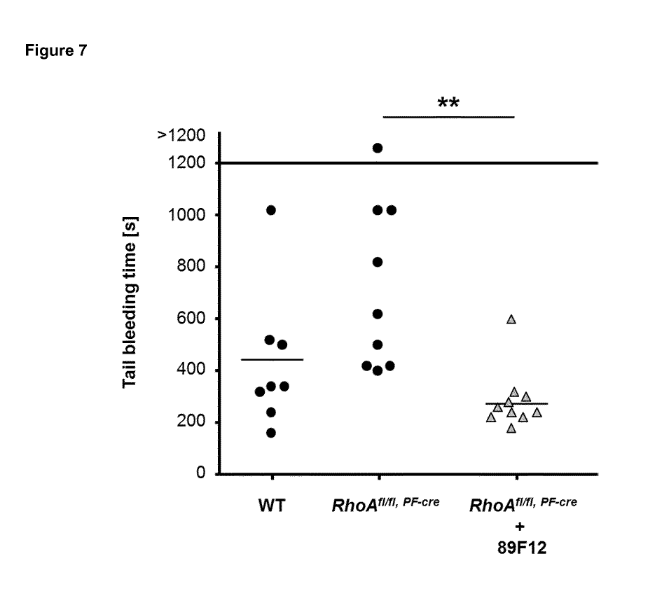

Megakaryocyte- and platelet specific RhoA-deficient mice displayed a pronounced macrothrombocytopenia with reduction of platelet counts by 50% (Pleines I, et al. Blood. 2012; 119: 1054-63). Lack of RhoA prolonged tail bleeding times (720±321 s, compared to 430±267 s in WT mice). In contrast, 89F12 treatment of RhoA-deficient mice could rescue the hemostatic defect of RhoA^{fl/fl, PF4-cre} mice resulting in shortened tail bleeding times compared to WT mice (286±118 s for 89F12-treated RhoA-deficient mice, P<0.01 compared to RhoA-deficient mice, FIG. 7). This indicated that antibody-mediated GPV-blockade can also compensate for the lack of critical downstream signaling molecules.

Thrombocytopenia is a critical risk factor for bleeding. To assess the role of GPV blockade under these conditions, thrombocytopenia was induced in mice by injection of two monoclonal anti-GPIbα antibodies, which deplete circulating platelets in mice independently of immune effector mechanisms (Nieswandt B, et al. Blood. 2000; 96: 2520-7; Bergmeier W, et al. Blood. 2000; 95: 886-93). Initial platelet counts were assessed as described above for each mouse and considered 100%. Subsequent counts were assessed 1 h after injection of the antibody and were normalized to the initial values. Wildtype mice with platelet count reduction below 15% of normal displayed prolonged bleeding times or were unable to stop the bleeding within the observation period of 20 min. In contrast, platelet count reductions up to 5% of normal did not alter tail bleeding times compared to 89F12-treated mice having a normal platelet count, but showed significantly shortened tail bleeding times when compared to platelet-depleted WT-mice indicating that the blockade of GPV lowers the platelet count required to maintain normal hemostasis (not shown).

### Materials and Methods

### Mice

C57BL/6JRj (Janvier Labs) were used as wildtype control mice. Mice lacking GPV (Gp5^{-/-}, the α2-integrin subunit (Itga2^{-/-} were described previously (Kahn M. et al., Blood 1999. 94: 4112-21; Holtkötter O. et al., J Biol Chem. 2002. 277(13): 10789-94). Mice lacking RhoA in megakaryocytes/platelets (RhoA^{fl/fl, Pf4-cre+/-}) were described previously (Pleines I. et al., Blood 2012. 119: 1054-63). GPVI was depleted by injecting 100 μg of the anti-GPVI antibody, JAQ1 (Nieswandt B, et al. J Exp Med. 2001; 193: 459-69.), i.p. 6 d before the experiment. CLEC-2 was depleted by i.p. injection of 200 μg INU1 ((May F, et al. Blood. 2009; 114: 3464-72); anti-CLEC-2 antibody). GPV blockade was achieved by injecting 50 μg 89F12-IgG or Fab-fragments 30 min to 12 h before the experiment. Animal studies were approved by the district government of Lower Franconia (Bezirksregierung Unterfranken).

### Monoclonal Antibodies

### Generation of Anti-GPV Antibodies

Female Wistar rats, 6 to 8 weeks of age, were immunized repeatedly with mouse platelets. The rat spleen cells were then fused with mouse myeloma cells (Ag14.653) and hybridomas were selected in HAT medium. Hybridomas secreting monoclonal antibodies (mAbs) directed against platelet surface antigens were identified by flow cytometry (see below). Gp5^{-/-} platelets were used as controls to test antibody specificity against GPV. Positive hybridomas were subcloned twice before large-scale production.

The further antibodies used are summarized in the following table.

| | Internal | | |
|---|---|---|---|
| Antibody | Name | Antigen | Described in |
| INU1 | 11E9 | CLEC-2 | [7] |
| JAQ1 | 98A3 | GPVI | [31] |
| p0p/B | 57E12 | GPIb | [33] |
| JON/A | 4H5 | GPIIb/IIIa | Bergmeier et al., Cytometry 2002 |

### Buffers and Media

All buffers were prepared with double-distilled water.

Phosphate buffered saline (PBS), pH 7.14

| | | |
|---|---|---|
| NaCl | 137 | mM |
| KCl | 2.7 | mM |
| KH₂PO₄ | 1.5 | mM |
| Na₂HPO₄ | 8 | mM |

### In Vitro Platelet Analyses

### Platelet Preparation and Washing

Mice were bled under isoflurane anesthesia from the retroorbital plexus. 700 μl blood were collected into a 1.5 ml reaction tube containing 300 μl heparin in TBS (20 U/ml, pH 7.3). Blood was centrifuged at 800 rpm (52 g; in a Eppendorf Centrifuge 5415C) for 5 min at RT. Supernatant and buffy coat were transferred into a new tube and centrifuged at 800 rpm for 6 min at RT to obtain platelet rich plasma (PRP). To prepare washed platelets, PRP was centrifuged at 2800 rpm (639 g) for 5 min at RT in the presence of prostacyclin (PGI₂) (0.1 μg/ml) and the pellet was resuspended in 1 ml Ca²⁺-free Tyrode's buffer containing PGI₂ (0.1 μg/ml) and apyrase (0.02 U/ml). After 10 min incubation at 37° C. the sample was centrifuged at 2800 rpm for 5 min. After resuspending the platelets once more in 1 ml Ca²⁺-free Tyrode's buffer, the platelet levels were determined taking a 1:10 dilution of the platelet solution and measuring platelet counts in a Sysmex counter (see below). The pellet was resuspended in the volume of Tyrode's buffer containing apyrase (0.02 U/ml) required to obtain 500,000 platelets/μl and left to incubate for at least 30 min at 37° C. before analysis. For determination of platelet count and size, 50 μl blood were drawn from the retroorbital plexus of anesthetized mice using heparinized microcapillaries and collected into a 1.5 ml reaction tube containing 300 μl heparin in TBS (20 U/ml, pH 7.3). The heparinized blood was diluted with PBS and platelet counts and size were determined using a Sysmex KX-21N automated hematology analyzer (Sysmex Corp., Kobe, Japan).

### Flow Cytometry

For determination of glycoprotein expression levels, platelets (1*10⁶) were stained for 10 min at RT with saturating amounts of fluorophore-conjugated antibodies described above and analyzed directly after addition of 500 μl PBS. The reaction was stopped by addition of 500 μl PBS and samples were analyzed on a FACSCalibur (Becton Dickinson, Heidelberg, Germany). For platelet activation studies, washed blood was incubated with the indicated agonists in the presence of JON/A-PE and anti-P-selectin-FITC for 15 min.

### ELISA

Washed platelets were prepared as described above and stimulated with 0.1 U/ml thrombin for 15 min at 37° C. Afterwards, the platelet suspension was centrifuged for 5 min at 2800 rpm, the supernatant was transferred to a new reaction tube and once again centrifuged for 5 min at maximal speed. 100 μl of the thrombin-stimulated platelet supernatant were transferred to a 96-well plate which was previously coated with 30 μg 89H11 (anti-GPV antibody) and blocked with 5% BSA/PBS. After an incubation of 1 h, the 96-well plate was washed and incubated with HRP-labelled 89F12, a second anti-GPV antibody, to detect the cleaved GPV. The HRP substrate was developed using TMB, the reaction was stopped with H₂SO₄ and developed in an ELISA reader at 405 nm.

### Platelet Aggregation

50 μl washed platelets (with a concentration of 0.5×10⁶ platelets/μl) or heparinized PRP was transferred into a cuvette containing 110 μl Tyrode's buffer (with 2 mM CaCl₂ and 100 μg/ml human fibrinogen). For determination of aggregation, antibodies were added to a final concentration of 10 μg/ml, agonists were added 100-fold concentrated and light transmission was recorded over 10 min on an Apact 4-channel optical aggregation system (APACT, Hamburg, Germany). For calibration of each measurement before agonist addition Tyrode's buffer was set as 100% aggregation and washed platelet suspension or PRP was set as 0% aggregation.

### In Vivo Analyses of Platelet Function

### Platelet Depletion

Initial platelet counts were assessed as described above for each mouse. These values were considered 100% and subsequent counts were normalized to these values. Mice were injected intravenously with 0.2 μg anti-GPIb antibodies per g mouse to induce an Fc-independent thrombocytopenia. Mice were injected 1 h before the experiment to achieve a remaining platelet count of 5-10% to induce FcγR-independent thrombocytopenia. 1 h after injection, platelet counts were again assessed.

### Mechanical Injury of the Abdominal Aorta

To open the abdominal cavity of anesthetized mice (10-16 weeks of age), a longitudinal midline incision was performed and the abdominal aorta was exposed. A Doppler ultrasonic flow probe (Transonic Systems, Maastricht, Netherlands) was placed around the aorta and thrombosis was induced by mechanical injury with a single firm compression (15 s) of a forceps upstream of the flow probe. Blood flow was monitored until complete occlusion occurred or 30 min had elapsed.

### Bleeding Time Assay

Mice were anesthetized by intraperitoneal injection of triple anesthesia and a 2-mm segment of the tail tip was removed with a scalpel. Tail bleeding was monitored by gently absorbing blood with filter paper at 20 s intervals without directly contacting the wound site. When no blood was observed on the paper, bleeding was determined to have ceased. The experiment was manually stopped after 20 min by cauterization.

### Statistical Analysis

Results are shown as mean±SD from at least three individual experiments per group. When applicable Fisher's exact test was used for statistical analysis. Otherwise, the Welch's t test was performed for statistical analysis. P-values<0.05 were considered statistically significant.

1. (canceled)

2. A method for the treatment or prevention of a hemorrhagic condition comprising administering to a subject in need thereof an effective amount of an inhibitor of platelet glycoprotein V (GPV).

3. The method according to claim 2, wherein the hemorrhagic condition is caused by a platelet disorder.

4. The method according to claim 3, wherein the platelet disorder is characterized by a decreased number of platelets.

5. The method according to claim 2, wherein the hemorrhagic condition is selected from the group consisting of inflammatory bleeding, hemorrhagic stroke, excessive bleeding due to sepsis, excessive bleeding due to thrombocytopenia, excessive bleeding due to disseminated intravascular coagulation (DIC), excessive bleeding due to chemotherapy, excessive bleeding due to hemolytic-uremic syndrome, excessive bleeding upon administration of soluble GPV, and excessive bleeding due to HIV infection.

6. The method according to claim 2, wherein the GPV is human GPV.

7. The method according to claim 2, wherein the inhibitor is an antibody directed against the extracellular domain of GPV, or a functional fragment or derivative of an antibody, the fragment or derivative being capable of binding to the extracellular domain of GPV.

8. The method according to claim 7, wherein the antibody, fragment, or derivative binds to a region of the extracellular domain of GPV which is different from the collagen-binding site of GPV.

9. The method according to claim 7, wherein the antibody, fragment, or derivative does not delay collagen-induced aggregation.

10. The method according to claim 7, wherein the antibody is a monoclonal antibody or a functional fragment or functional derivative thereof.

11. The method according to claim 2, wherein the inhibitor is a nucleic acid capable of reducing expression of GP5 mRNA.

12. The method according to claim 2, wherein the inhibitor, upon administration to the subject, does not affect the number of platelets in the subject.

13. The method according to claim 2, wherein the inhibitor is a coagulant.

14. The method according to claim 2, wherein the treatment or prevention comprises administering to a subject, a pharmaceutically effective amount of the inhibitor.

15. The method according to claim 14, wherein the treatment or prevention further comprises administering to the subject a coagulant other than the inhibitor.

16. The method according to claim 15, wherein the coagulant other than the inhibitor is selected from the group consisting of an anti-fibrinolytic agent, a platelet concentrate, a coagulation factor concentrate, and fresh frozen plasma.

17. A pharmaceutical composition comprising an inhibitor of platelet glycoprotein V (GPV) and a pharmaceutically acceptable excipient.

18. (canceled)

19. A method of treating or preventing a hemorrhagic condition in a subject, comprising administering to the subject an effective amount of the pharmaceutical composition of claim 17.

20. The method of claim 14, wherein the subject is a human.

21. The method of claim 19, wherein the subject is a human.
